(19) United States
(12) Patent Application Publication
(10) Pub. No.: US 2008/0295602 A1
(43) Pub. Date: Dec. 4, 2008

(54) Method and System for Sorting Green Lumber

(76) Inventor: Gavin Wallace, Wainuiomata (NZ)

Correspondence Address: LEWIS, RICE & FINGERSH, LC;ATTN: BOX IP DEPT., 500 NORTH BROADWAY, SUITE 2000, ST LOUIS, MO, 63102, US

(21) Appl. No.: 12/128,813

(22) Filed: May 29, 2008

(30) Foreign Application Priority Data
Jun. 1, 2007 (NZ) 555606

(51) Int. Cl. G01N 33/46; G01M 3/24; G01N 3/30

(52) U.S. Cl. 73/602; 700/214; 144/356; 73/73; 73/584; 702/56

(57) ABSTRACT

A system for sorting green lumber into groups for drying includes a striking device arranged to provide an impact on one end of a piece of lumber. A vibration sensor is arranged to detect vibrations in the piece of lumber caused by the impact on the piece of lumber by the striking device. A computation device is configured to determine the acoustic velocity of the piece of lumber from the detected vibrations and a length of the piece of lumber. An indication device provides an indication of a one of the drying groups for the piece of lumber based on the determined acoustic velocity. Alternatively, the indication device may be a bin sorter that receives a signal from the computation device and sorts the lumber into a one of the groups in response to the signal, for example.

### BACKGROUND OF THE INVENTION

1. Field of the Invention

This present invention relates to a method and system for sorting green lumber for drying.

2. Description of Related Art

The objectives in kiln drying of lumber include to dry each piece of lumber as economically as possible to the same moisture content and without incurring any degradation in the quality of the wood. With many softwoods, it has been found that lumber sorted according to green density can be grouped into charges and, with different drying regimes applied to each charge, kiln drying can be optimised for cost savings, wood quality and wood recovery. However, hard pines, such as the softwood class collectively known as southern yellow pine, have a very narrow distribution in green density, and kiln drying optimisation based on a green density sort is not viable.

The dynamic modulus of elasticity of a piece of sawn lumber is defined as the product of the density of the lumber and the square of the velocity of an acoustic wave transmitted longitudinally within the lumber.

### SUMMARY

The following is a summary of the invention in order to provide a basic understanding of some of the aspects of the invention. This summary is not intended to identify key or critical elements of the invention or to delineate the scope of the invention. The sole purpose of this section is to present some concepts of the invention in a simplified form as a prelude to the more detailed description that is presented later.

It is an object of at least a preferred embodiment of the present invention to provide a system and method for the sorting of softwood hard pine lumber into separate groups or kiln charges to enable optimisation of the drying process, or at least to provide the public with a useful choice.

The present invention provides a method of sorting pieces of green lumber into groups for drying comprising: providing an impact on one end of a piece of lumber; detecting vibrations in the piece of lumber caused by the impact; determining the acoustic velocity of the piece of lumber from the detected vibrations and a length of the piece of lumber; and grouping the piece of lumber according to the determined acoustic velocity prior to drying.

Preferably, providing an impact on one end of the piece of lumber includes striking the one end of the piece of lumber with a striking device. The striking device may be a hammer, for example.

Preferably, the method further comprises detecting the presence of lumber, and providing the impact on one end of the lumber in response to detecting the presence of the piece of lumber. Detecting the presence of lumber may include detecting lumber breaking a light beam, for example.

Preferably, detecting vibrations in the piece of lumber includes detecting vibrations in the piece of lumber at one of the ends of the piece of lumber.

Preferably, the method further comprises digitally recording the vibrations detected in the piece of lumber, and determining the acoustic velocity of the piece of lumber includes Fourier transforming the digitally recorded vibrations to determine the resonant frequency of the vibrations in the lumber. The method may further comprise filtering the digitally recorded vibrations before Fourier transforming the filtered vibrations.

The method may further comprise determining the length of the piece of lumber. Determining the length of the piece of lumber may include measuring the length of the piece of lumber using light, for example. Alternatively, determining the length of the piece of lumber may include measuring the length of the piece of lumber mechanically using a mechanical device, for example.

Alternatively or additionally, the method may further comprise cutting the piece of lumber to a known length before providing the impact on one end of the piece of lumber.

Preferably, the method further comprises assessing a drying regime for each group of lumber based on acoustic velocity(s) of lumber in the group.

Preferably, the drying is kiln drying.

The present invention further provides a system for sorting green lumber into groups for drying comprising: a striking device arranged to provide an impact on one end of a piece of lumber; a vibration sensor arranged to detect vibrations in the piece of lumber caused by the impact on the piece of lumber by the striking device; a computation device configured to determine the acoustic velocity of the piece of lumber from the detected vibrations and a length of the piece of lumber; and an indication device for providing an indication of a one of the drying groups for the piece of lumber based on the determined acoustic velocity.

The striking device may be a hammer, for example.

Preferably, the system further comprises a device to detect the presence of the piece of lumber, the device to detect the presence of the piece of lumber being arranged to trigger operation of the striking device in response to detecting a piece of lumber. The device to detect the presence of lumber may be arranged to detect lumber breaking a light beam, for example.

Preferably, the vibration sensor is arranged to detect vibrations in the piece of lumber at one of the ends of the piece of lumber. The vibration sensor may be a microphone, for example.

Preferably, the system is arranged to digitally record the detected vibrations, and the computation device is configured to Fourier transform the digitally recorded vibrations to determine the resonant frequency of the vibrations in the piece of lumber. Preferably, the computation device is configured to filter the digitally recorded vibrations before Fourier transforming the filtered vibrations.

Preferably, the system further comprises length measuring apparatus for measuring the length of the piece of lumber. The length measuring apparatus may be arranged to measure the length of the piece of lumber using light, for example. Alternatively, the length measuring apparatus may be a mechanical device arranged to measure the length of the piece of lumber mechanically, for example.

Alternatively or additionally, the system may further comprise cutting apparatus for cutting the piece of lumber to a known length before providing the impact on one end of the piece of lumber.

Preferably, the computation device is configured to determine a drying regime for each group of lumber based on acoustic velocity(s) of lumber in the group.

The present invention further provides a method of sorting pieces of green lumber into groups for drying comprising: providing an impact on one end of a piece of lumber; detecting vibrations in the piece of lumber caused by the impact; determining the acoustic velocity of the piece of lumber from the detected vibrations and a length of the piece of lumber; determining the modulus of elasticity of the piece of lumber from the determined acoustic velocity and a density of the piece of lumber; and sorting the piece of lumber into one of the drying groups based on the modulus of elasticity of the lumber.

Preferably, the method further comprises assessing the attenuation of gamma rays passing through at least one area of the piece of lumber, and determining the density of the piece of lumber from the attenuation of the gamma rays and a thickness of the piece of lumber. Preferably, the method further comprises assessing the attenuation of gamma rays passing through two or more areas of the piece of lumber.

Alternatively, the density may be determined by determining the weight and the thickness, length and width of the piece of lumber, and determining the density of the piece of lumber based on the weight and volume of the piece of lumber, for example.

Preferably, the method further comprises measuring the thickness of the piece of lumber.

Alternatively or additionally, the method may further comprise cutting the piece of lumber to a known thickness before assessing the attenuation of gamma rays passing through at least one area of the piece of lumber.

Preferably, the method further comprises assessing a drying regime for each group of lumber based on modulus(s) of elasticity of lumber in the group.

The present invention further provides a method of sorting green lumber conveyed on a conveyor system into groups for drying comprising: assessing the attenuation of gamma rays passing through at least one area of a piece of lumber passing between a gamma ray source and detector; determining the thickness of the piece of lumber as the piece of lumber passes thickness determining apparatus; providing an impact on one end of the piece of lumber; detecting vibrations in the piece of lumber caused by the impact as the piece of lumber passes a vibration sensor; determining the length of the piece of lumber as the piece of lumber passes length determining apparatus; determining the acoustic velocity of the piece of lumber from the detected vibrations and the length of the piece of lumber; determining the density of the piece of lumber from the attenuation of the gamma rays and the thickness of the piece of lumber; determining the modulus of elasticity of the piece of lumber from the determined acoustic velocity and the determined density of the piece of lumber; and sorting the piece of lumber into one of the drying groups based on the modulus of elasticity of the lumber.

The present invention still further provides a system for sorting green lumber into groups for drying comprising: a striking device arranged to provide an impact on one end of the piece of lumber; a vibration sensor arranged to detect vibrations in the piece of lumber caused by the impact on the piece of lumber by the striking device; a computation device configured to determine the acoustic velocity of the piece of lumber from the detected vibrations and a length of the piece of lumber, the computation device being configured to determine the modulus of elasticity form the determined acoustic velocity and a density of the piece of lumber; and an indication device for providing an indication of a one of the drying groups for the piece of lumber based on the determined modulus of elasticity.

Preferably, the system further comprises a gamma ray device arranged to pass gamma rays through at least one area of a piece of lumber and record the attenuation of the gamma rays passed through the piece of lumber, wherein the computation device is further configured to determine the density of the piece of lumber from the attenuation of the gamma rays and a thickness of the piece of lumber.

Preferably, the gamma ray device is arranged to pass gamma rays through two or more areas of the piece of lumber and record the attenuation of the gamma rays passed through the piece of lumber.

Preferably, the system further comprises thickness measuring apparatus for measuring the thickness of the piece of lumber.

Alternatively or additionally, the system may further comprise cutting apparatus for cutting the piece of lumber to a known thickness before passing gamma rays through at least one area of a piece of lumber and recording the attenuation of the gamma rays passed through the piece of lumber.

Preferably, the computation device is configured to provide a drying regime for each group of lumber based on modulus(s) of elasticity of lumber in the group.

To those skilled in the art to which the invention relates, many changes in construction and widely differing embodiments and applications of the invention will suggest themselves without departing from the scope of the invention as defined in the appended claims. The disclosures and the descriptions herein are purely illustrative and are not intended to be in any sense limiting. Where specific integers are mentioned herein which have known equivalents in the art to which this invention relates, such known equivalents are deemed to be incorporated herein as if individually set forth.

As used herein the term “and/or” means “and” or “or”, or both.

As used herein “(s)” following a noun means the plural and/or singular forms of the noun.

### BRIEF DESCRIPTION OF THE DRAWINGS

The invention will be further described by way of example only and without intending to be limiting, with reference to the following drawings, wherein:

FIG. 1 is a system for sorting green lumber into groups for drying based on acoustic velocity according to a first embodiment of the invention.

FIG. 2 is a block diagram showing the components of the system shown in FIG. 1;

FIGS. 3A and 3B are sound recordings of vibrations in two samples of 100×50 mm lumber.

FIGS. 4A and 4B are frequency spectrums of the sound recordings of FIGS. 3A and 3B respectively.

FIG. 5 shows the distribution of acoustic velocity as measured in a trial of 100 southern yellow pines boards.

FIG. 6 shows the kiln drying results of a southern yellow pine trial.

FIG. 7 is a block diagram showing components of a system for sorting green lumber into groups for drying based on modulus of elasticity according to a second embodiment of the invention.

### DETAILED DESCRIPTION OF THE PREFERRED EMBODIMENT(S)

The following detailed description illustrates by way of example and not by way of limitation.

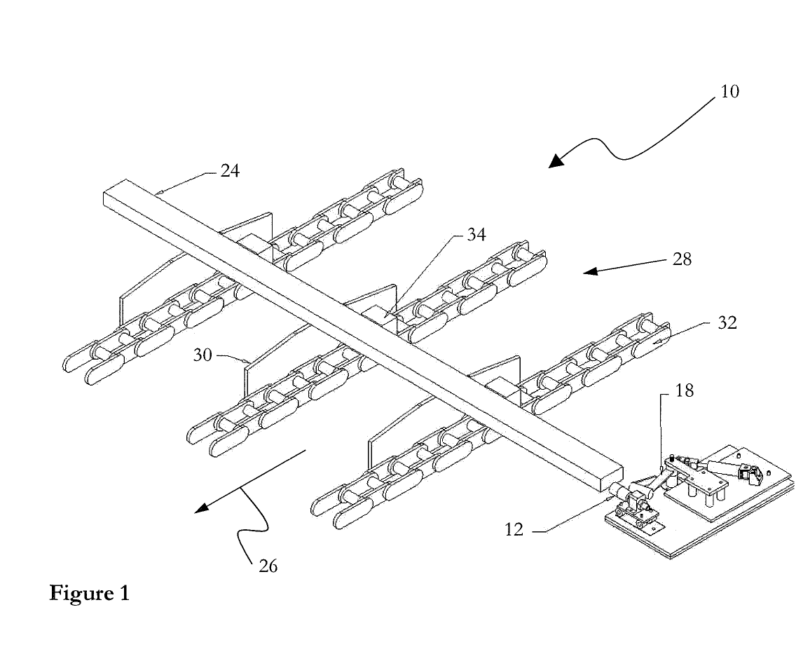

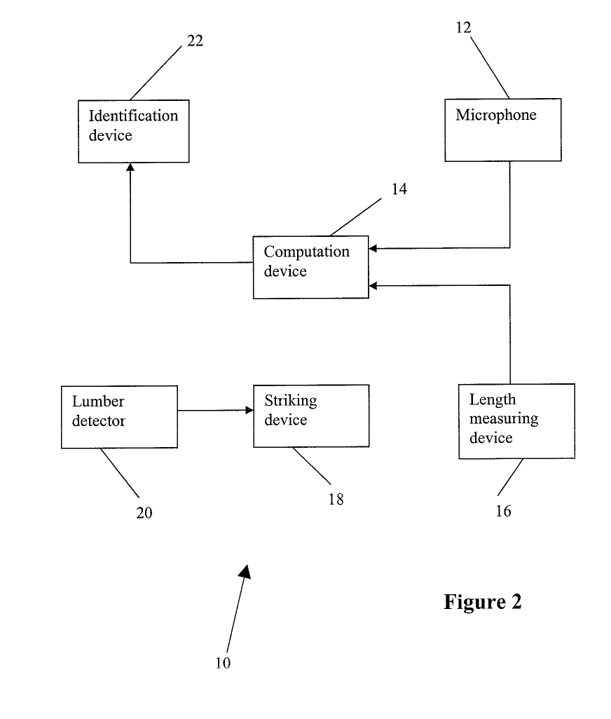

FIG. 1 shows part of a system 10 for sorting green lumber into groups for drying according to a first embodiment of the invention. FIG. 2 is a block diagram showing the components of the system 10 used to determine the longitudinal acoustic velocity. The system 10, shown schematically in FIG. 2, includes vibration sensor in the form of microphone 12, computation device 14, length measuring apparatus or device 16, striking device 18, lumber detector 20, and indication or identification device 22. The computation device 14 will typically communicate with a bin sorter programmable logic controller, or will directly control a paint spraying system to implement labelling and manual sorting of green lumber pieces or boards, for example.

The system 10 may be used to sort green hard pine lumber, such as southern yellow pine, for example. By determining the acoustic velocity of the green hard pine lumber before drying, the optimum drying regime can be established, and the kiln-dried moisture content can be better controlled during drying.

With reference to FIG. 1, lumber pieces or boards 24 are aligned transverse to their direction of travel, indicated by arrow 26, on a chain conveyor system 28, for example. Thin shaped plates 30 support each lumber piece 24 above the chain links 32, with the lumber piece 24 maintaining contact with driving lugs 34 of the system 28. The ends of the pieces of lumber 24 may be aligned to the striking device 18 by previously moving the pieces of lumber 24 to a fence (not shown).

The acoustic velocity in the lumber 24 is measured using the microphone 12, striking device 18 and length measuring device 16. Striking device 18 is used to provide an impact on one end of the lumber 24. The presence of the lumber 24 within the range of the striking device 18 is detected by lumber detector 20 (not shown in FIG. 1). The lumber detector 20 may be any detector that can detect the presence of the lumber 24. In one embodiment, the lumber detector 20 is a light source and light detector where lumber between the source and detector is detected by the lack of light at the light detector. In other embodiments, different lumber detectors can be used.

Once the lumber 24 is detected, the lumber detector 20 provides a signal to the striking device 18. Typically the signal will be an electrical signal. Upon receipt of the signal, the striking device 18 strikes one end of the lumber 24. In one embodiment, the striking device 18 is a hammer. In other embodiments, any blunt instrument may be used. The action of striking the lumber 24 sets up a longitudinal acoustic wave within the lumber 24.

Microphone 12 is used to detect the acoustic wave within the lumber 24. In preferred embodiments, microphone 12 is positioned at one of the ends of the lumber. This end may be either the end with the striker device 18 as shown in FIG. 1 or the end opposite the striker device 18. The acoustic wave detected by the microphone 12 is then converted to a digital signal. The conversion into a digital signal may take place at either the microphone 12 or the computation device 14.

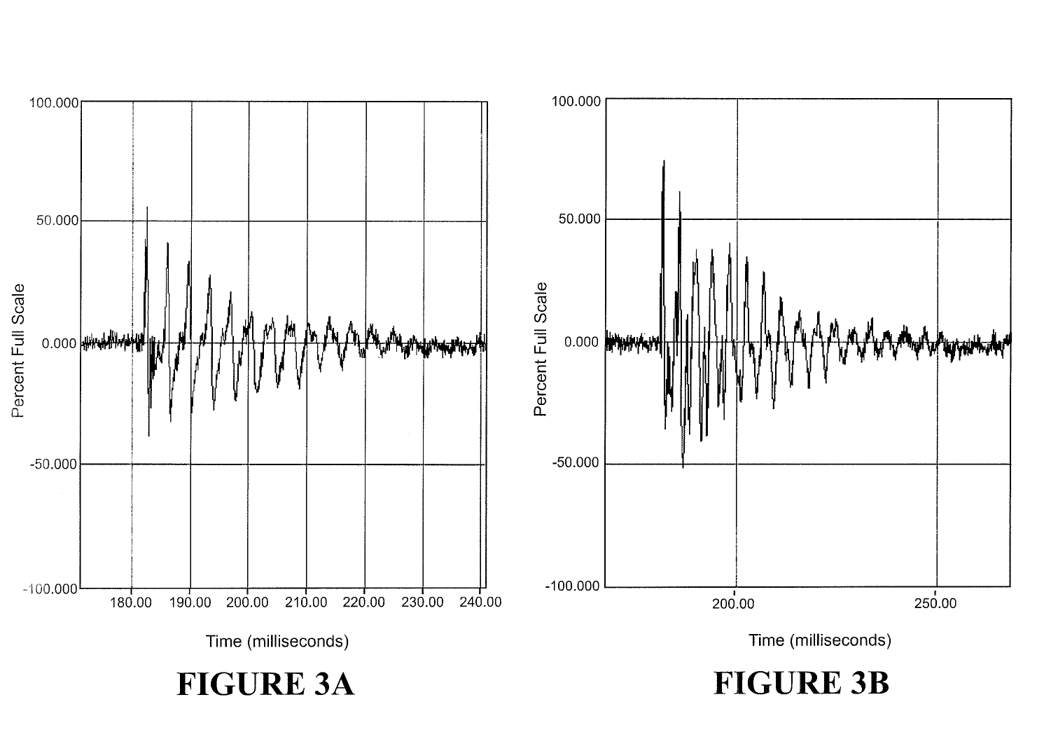

FIGS. 3A and 3B show examples of wavelengths in two pieces of green 100×50 mm lumber recorded with a microphone 12 after the pieces of lumber have been struck with a striking device 18. The left hand sides of FIGS. 3A and 3B shows the background noise before the acoustic wave impacted on the microphone 12. The acoustic wave then echoed through the lumber and produced the decaying images of FIGS. 3A and 3B.

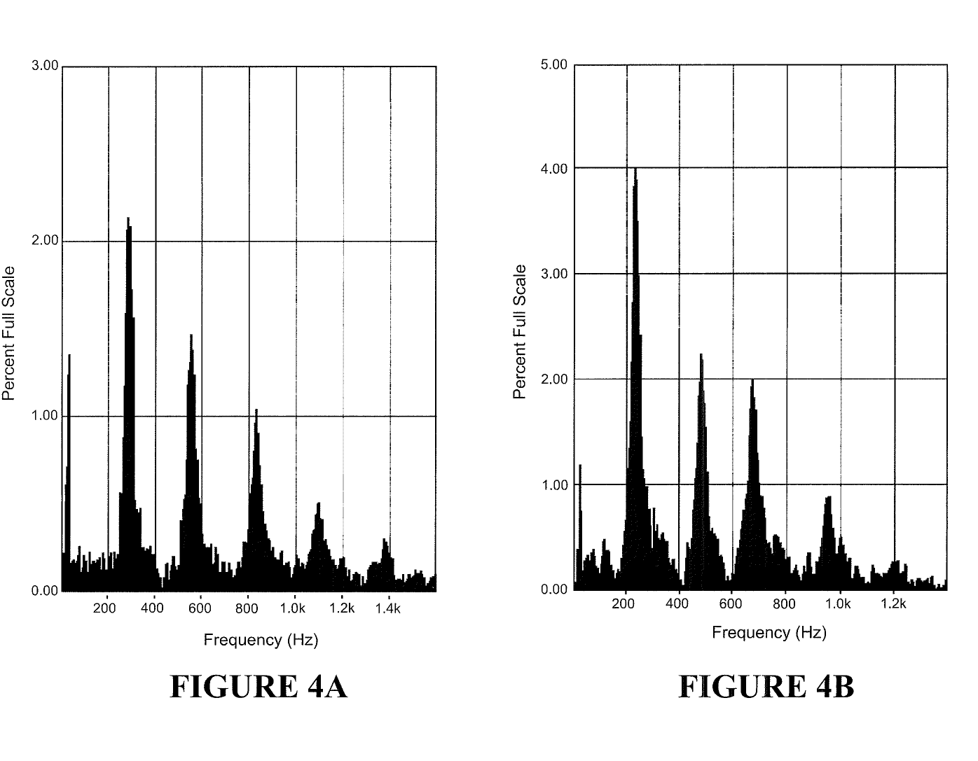

After measurement with the microphone 12, the measured signals are digitised in an A/D converter. The signals may also be filtered to remove unwanted noise from the same. Following digitisation and filtering, the signals are transformed from time based signals to frequency based signals using a Fourier transform. The Fourier transformed images of FIGS. 3A and 3B are shown in FIGS. 4A and 4B respectively. Both of FIGS. 4A and 4B clearly show a resonant peak and harmonics of the acoustic velocity wave in the lumber.

The wavelength of the resonance modes of a piece of lumber of length L are

$$\begin{matrix}{λ}_{n} = \frac{2 L}{n} & n = 1 , 2 , 3 , …\end{matrix} \qquad 1$$

where both ends of the lumber are free. The corresponding resonant frequencies are

$${f}_{n} = \frac{v}{{λ}_{n}} \qquad 2$$

where v is the acoustic longitudinal velocity. Because of secondary effects, the base frequency fl can be perturbed and it is preferable to use an average of the harmonics of the base frequency to calculate the base frequency fl. As can be seen in FIGS. 4A and 4B, the harmonics above the fundamental frequency are clearly distinguishable and easy to measure.

Referring back to FIG. 2, as the lumber 24 moves along the conveyor system 28 it passes the length measuring device 16 (not shown in FIG. 1). Length measuring device 16 measures the length of the lumber 24. The length may be measured nominally using either light or mechanical sensors, or may be measured more precisely using laser or transducer sonic range finders. The length of each piece of lumber 24 may be measured before or after, or substantially at the same time as, striking the lumber 24 and measuring the longitudinal acoustic wave. That is, the length measuring device 16 may be located upstream or downstream of, or generally in the same location as, the microphone 12 and striking device 18 on the conveyor system 28, for example.

Alternatively or additionally, cutting apparatus (not shown) may be used to cut each piece of lumber 24 to a one of one or more predetermined nominal lengths before striking one end of the piece of lumber with the striking device.

Alternatively or additionally, the length measuring device 16 may include a plurality of detectors that provide a nominal length indication for each piece of lumber 24, for example. In one embodiment, the detectors are photocells. Five photocells and associated detectors may be used to provide an indication of the nominal length of each piece of lumber 24, for example. However, any suitable number of photocells may be used; for example, in some applications seven photocells may be used.

The measured or nominal length measurement of the piece of lumber 24 is provided to computation device 14 for calculating the acoustic velocity of the lumber 24.

The computation device 14 can determine the acoustic longitudinal velocity from the length measurement of the lumber 24, the wavelengths of at least one resonant mode and at least one resonant frequency.

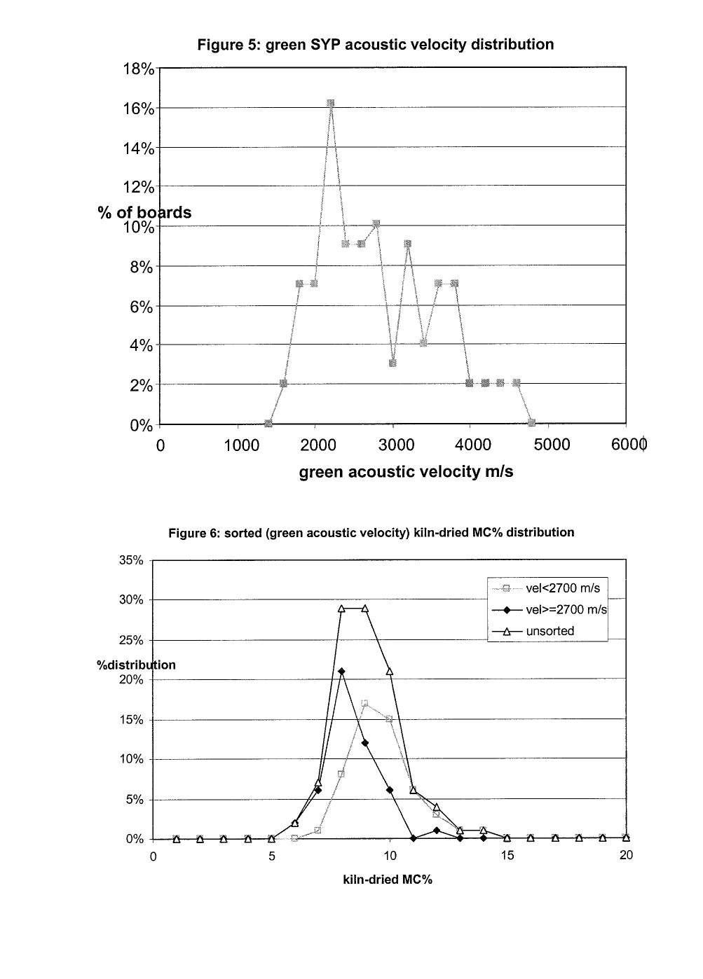

The two pieces of lumber subject to measurement in FIGS. 3A, 3B, 4A, and 4B were 6 metres long and had acoustic velocities of 3288 and 2856 m/s. The typical distribution of acoustic velocities of 100 pieces of southern yellow pine lumber is shown in FIG. 5.

The computation device 14 may be further arranged to determine a drying regime that should be applied to the lumber 24. The example southern yellow pine pieces depicted in FIG. 5 were kiln dried under the same conditions, and their subsequent moisture contents measured. The mean kiln-dried moisture content (MC) was found to be 8.6% (standard deviation 1.40%), and the moisture was spread over a range of 5.5% to 13.9%. When the boards were sorted into two groups using a threshold green acoustic velocity of 2700 m/s, the mean moisture content for the low velocity group was 9.2% (standard deviation 1.31%), and 7.9% (1.12%) for the high velocity group. This separation is shown in FIG. 6. Clearly, the high velocity group required less drying time, and a reduction in spread of the kiln-dried moisture contents could be achieved.

The computation device 14 communicates with the indication or identification device 22 (not shown in FIG. 1) for providing an indication of (or identifying) the sorting or drying regime for each piece of lumber 24. The indication device 22 may be a paint spraying system that sprays a visual indication on the lumber 22 indicating the drying regime for the lumber 24, for example. In a two-sort system, the paint spraying system may spray only lumber for one drying regime and not the other, for example. That is, the indication device 22 may provide an indication that a first piece of lumber 24 belongs to a lower velocity drying group by spraying the first piece of lumber 24, and the indication device 22 may provide an indication that a second piece of lumber 24 belongs to a higher velocity drying group by not spraying the second piece of lumber 24.

In alternative embodiments, the indication device 22 may be a bin sorter that receives a signal from the computation device 14 and sorts the lumber 24 into groups in response to the signal. That is, the indication device 22 may provide an indication (or identifies) that a first piece of lumber 24 belongs to a lower velocity drying group by sorting or segregating the first piece of lumber 24 to the lower velocity group, and the indication device 22 may provide an indication (or identifies) that a second piece of lumber 24 belongs to a higher velocity drying group by sorting or segregating the second piece of lumber 24 to the higher velocity group.

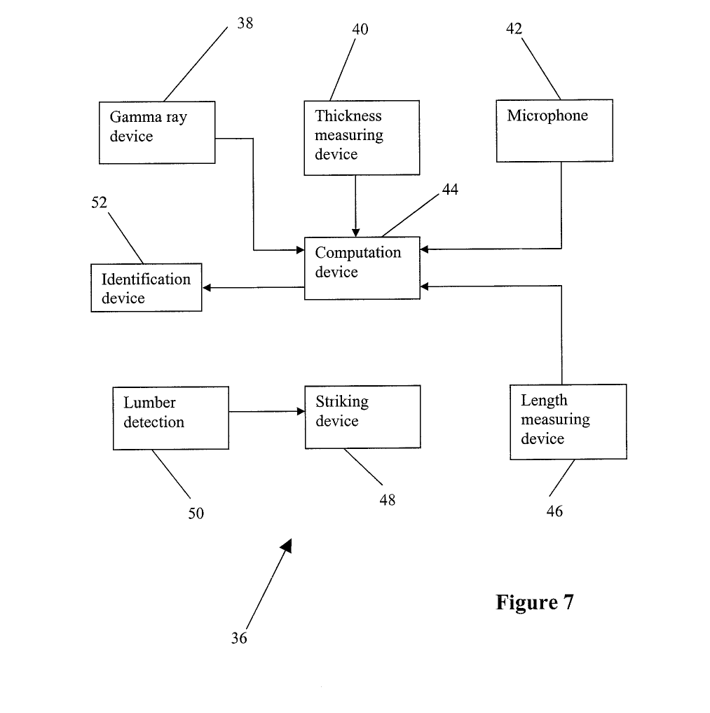

FIG. 7 is a block diagram showing the components of a system 36 for sorting green lumber into groups for drying according to a second embodiment of the invention. The system 36 sorts pieces of lumber into drying groups based on the modulus of elasticity of the lumber. The system 36, shown schematically in FIG. 7, includes a gamma ray device 38, thickness measuring apparatus or device 40, microphone 42, computation device 44, length measuring apparatus or device 46, striking device 48, lumber detector 50, and indication or identification device 52.

The dynamic modulus of elasticity of a piece of lumber is defined as the product of the density of the lumber and the square of the velocity of an acoustic wave within the lumber. The modulus of elasticity can be used to predict the strength of the green lumber after drying. Typically, the green lumber is cut to its final size and then the modulus of elasticity is determined before the lumber is dried.

The present inventor has determined that the modulus of elasticity can also be used to sort lumber into groups before drying to enable improved drying regimes and better moisture control when drying in kilns. By determining the modulus of elasticity of the green lumber before drying, the lumber can be graded and processed as necessary for its final purpose.

With reference to FIG. 7, in one embodiment, lumber moves along a conveyor belt system, as previously described with reference to FIGS. 1 and 2. The lumber passing along the conveyor belt system (not shown in FIG. 7) may comprise lumber of differing lengths and cross sections.

To determine the modulus of elasticity both the density of the lumber and the velocity of an acoustic wave within the lumber need to be determined.

The density of a piece of lumber 24 may be determined using gamma ray device 38 and thickness measuring device 40. The lumber 24 passes between a source of gamma rays and a gamma ray detector forming part of the gamma ray device 38. As the lumber 24 passes through the gamma ray device 38 the gamma rays at the detector are measured. These gamma rays are attenuated by the lumber 38 and the amount of attenuation is related to the amount of lumber 24 being scanned.

As the lumber 24 moves along the conveyor system it passes the thickness measuring device 46. The thickness measuring device 40 measures the thickness of the lumber 24. In one embodiment, laser beams are used to measure the thickness of the lumber 24. In other embodiments, the thickness may be measured by other means; for example, the thickness of the lumber 24 may be measured mechanically.

Alternatively or additionally, cutting apparatus (not shown) may be used to cut each piece of lumber 24 to a predetermined nominal thickness before assessing the attenuation of gamma rays passing through the lumber 24.

The green density of the lumber can be calculated from:

$${ρ}_{g} = - [ B + ln ( \frac{I}{{I}_{0}} ) / x ] / A \qquad 1$$

where ρg is the green lumber density, A and B are constants, x is the measured or nominal thickness of the lumber, I is the intensity of the gamma rays after passing through the lumber and I0 is the intensity of the gamma rays when no lumber is present. The constants are determined from calibrating the gamma ray device 38 for the particular application. These constants vary with different gamma ray devices and for different applications.

Preferably the attenuation of gamma rays passing through two or more areas of each piece of lumber 24 is measured and the results averaged. For example, four gamma ray beams may be spaced along the length of 6 metre long boards. Boards of lumber 24 can run from heart to sap wood along their length, and the green density can correspondingly change from one end to the other. Averaging of results provides a better estimation of the overall green density.

As the lumber 24 moves along the conveyor system it passes the length measuring device 46. The length measuring device 46 may include a plurality of detectors that provide a nominal length indication. In one embodiment, the detectors are photocells. For example, five photocells and associated detectors may be used to provide an indication of the nominal length of the lumber 24. However, any suitable number of photocells may be used; for example, in some applications seven photocells may be used.

Alternatively or additionally, as described above with reference to FIGS. 1 and 2, the length of the lumber 24 may be measured nominally using either light or mechanical sensors, or may be measured more precisely using laser or transducer sonic range finders. Alternatively or additionally, as described above with reference to FIGS. 1 and 2, each piece of lumber 24 may be cut to a one of one or more predetermined, known nominal lengths. The lumber may be cut to a known nominal length before or after cutting the lumber to a known nominal thickness, for example.

Alternatively, the green lumber density of each piece of lumber may be estimated by determining the weight, thickness, length and width of each board, and determining the density of the piece of lumber based on the weight and volume of the lumber, for example.

The lumber detector 50 detects the presence of lumber 24 on the conveyor system. Any suitable device may be used to detect the presence of lumber 24 on the conveyor system. In one embodiment, the lumber detector 50 may include a light source and light detector where lumber between the source and detector is detected by the lack of light at the light detector. In other embodiments different lumber detectors can be used. For example, the lumber detector 50 may include a microswitch to detect the presence of lumber 24 on the conveyor system. A shaft encoder may be used to record the relative movement of the board to determine width of the board and relation to density measurement. The shaft encoder may be used to correlate density and sound measurements.

Once the lumber 24 is detected, the lumber detector 50 provides a signal to the striking device 48. Typically the signal will be an electrical signal. Upon receipt of the signal, the striking device 48 strikes one end of the lumber 24. In one embodiment, the striking device 48 is a hammer. In other embodiments, any blunt instrument may be used. The action of striking the lumber 24 sets up an acoustic wave within the lumber 24. In preferred embodiments the signal to the striking device 48 is provided so that the striking device 48 strikes each piece of lumber 24 in the same relative place. For example, the signal from the lumber detector 50 may be timed so that the striking device 48 always strikes the lumber 24 at one edge of the lumber 24.

In preferred embodiments, information from the length detector 50 and thickness detector 40 may be processed to provide a force parameter to the striking device 48. This allows the force of the striking device 48 to be varied and different force impacts to be provided to lumber 24 of different weights. This variance can be used to prevent the striking device 48 providing a force to the lumber 24 that excessively shifts the lumber 24 on the conveyor system.

The microphone 42 is used to detect the acoustic wave within the lumber 24. In preferred embodiments, microphone 42 is positioned at one end of the lumber 24. This end may be either the end with the striker 48 or the end opposite the striker 48. As the lumber 24 moves past the microphone 42 on the conveyor system, the microphone 42 detects any acoustic waves in the lumber 24. In preferred embodiments, the microphone 42 records acoustic waves from the lumber 24 for a plurality of vibrations within the lumber 24. For example, the microphone 42 may be adapted to record for 50 milliseconds for each piece of lumber. The acoustic wave detected by the microphone 42 is then converted to a digital signal. The conversion into a digital signal may take place at either the microphone 42 or the computation device 44.

The computation device 44 is able to calculate the velocity of an acoustic wave within the lumber, as described with reference to FIGS. 3A, 3B, 4A, and 4B above, and using the calculated velocity to calculate the modulus of elasticity.

The computation device 44 may further be arranged to determine a drying regime that should be applied to the lumber 24 based on modulus of elasticity, similar to sorting and drying based on acoustic velocity described above with reference to FIGS. 1 to 6. The lumber 24 can be sorted into two groups using a threshold modulus of elasticity for example, with the higher modulus of elasticity group requiring less drying time and the lower modulus of elasticity group requiring more drying time. By sorting lumber 24 into two or more groups based on modulus of elasticity prior to drying, a reduction in spread of kiln-dried moisture contents of dried lumber can be achieved.

The computation device 44 communicates with the indication or identification device 52. The indication device 52 provides an indication of (or identifies) the sorting or drying regime for each piece of lumber 24. The indication device 54 may be a paint spraying system that sprays an indication on the lumber 24 indicating the drying regime for the lumber 24. In a two-sort system the paint spraying system may spray only lumber 24 for one drying regime and not the other.

In alternative embodiments, the indication device 52 may be a bin sorter that receives a signal from the computation device 44 and sorts the lumber 24 into drying groups in response to the signal.

It should be noted that the arrangements described above are embodiments only and are not intended to be limiting. The described embodiments relate to the use of the invention in an on-the-line application where measurements are preformed as lumber moves along a conveyor system. In these embodiments, the lumber is not stopped at any stage of the process. For example, in an on-the-line system, the lumber may pass the striker etc at a rate of one hundred and twenty boards per minute or even faster. An embodiment of the invention is arranged to work at the speed of the conveyor, where the speed of the conveyor is set by other requirements in the lumber processing plant. Further, the gamma ray sources and detectors in an embodiment of the present invention could be positioned at any suitable location along the conveyor system. For example, the gamma ray sources and detectors could be positioned either before or after the microphone and the striking device.

The term “comprising” as used in this specification and claims means “consisting at least in part of”. That is to say, when interpreting statements in this specification and claims which include “comprising”, the features prefaced by this term in each statement all need to be present but other features can also be present. Related terms such as “comprise” and “comprised” are to be interpreted in a similar manner.

While the invention has been disclosed in conjunction with a description of certain embodiments, including those that are currently believed to be the preferred embodiments, the detailed description is intended to be illustrative and should not be understood to limit the scope of the present disclosure. As would be understood by one of ordinary skill in the art, embodiments other than those described in detail herein are encompassed by the present invention. Modifications and variations of the described embodiments may be made without departing from the spirit and scope of the invention.

1. A method of sorting pieces of green lumber into groups for drying comprising:
providing an impact on one end of a piece of lumber;
detecting vibrations in the piece of lumber caused by the impact;
determining the acoustic velocity of the piece of lumber from the detected vibrations and a length of the piece of lumber; and
grouping the piece of lumber according to the determined acoustic velocity prior to drying.

2. The method of claim 1, wherein providing an impact on one end of the piece of lumber includes striking the one end of the piece of lumber with a striking device.

3. The method of claim 1, comprising:
detecting the presence of lumber; and
providing the impact on one end of the lumber in response to detecting the presence of the piece of lumber.

4. The method of claim 1, wherein detecting vibrations in the piece of lumber includes detecting vibrations in the piece of lumber at one of the ends of the piece of lumber.

5. The method of claim 1, comprising:
digitally recording the vibrations detected in the piece of lumber; and
determining the acoustic velocity of the piece of lumber includes Fourier transforming the digitally recorded vibrations to determine the resonant frequency of the vibrations in the lumber.

6. The method of claim 1, comprising:
determining the length of the piece of lumber.

7. The method of claim 1, comprising:
cutting the piece of lumber to a known length before providing the impact on one end of the piece of lumber.

8. The method of claim 1, comprising:
assessing a drying regime for each group of lumber based on acoustic velocity(s) of lumber in the group.

9. A system for sorting green lumber into groups for drying comprising:
a striking device arranged to provide an impact on one end of a piece of lumber;
a vibration sensor arranged to detect vibrations in the piece of lumber caused by the impact on the piece of lumber by the striking device;
a computation device configured to determine the acoustic velocity of the piece of lumber from the detected vibrations and a length of the piece of lumber; and
an indication device for providing an indication of a one of the drying groups for the piece of lumber based on the determined acoustic velocity.

10. The system of claim 9, comprising a device to detect the presence of the piece of lumber, the device to detect the presence of the piece of lumber being arranged to trigger operation of the striking device in response to detecting a piece of lumber.

11. The system of claim 9, wherein the vibration sensor is arranged to detect vibrations in the piece of lumber at one of the ends of the piece of lumber.

12. The system of claim 9, wherein the system is arranged to digitally record the detected vibrations, and the computation device is configured to Fourier transform the digitally recorded vibrations to determine the resonant frequency of the vibrations in the piece of lumber.

13. The system of claim 9, comprising:
length measuring apparatus for measuring the length of the piece of lumber.

14. The system of claim 9, comprising:
cutting apparatus for cutting the piece of lumber to a known length before providing the impact on one end of the piece of lumber.

15. The system of claim 9, wherein the computation device is configured to determine a drying regime for each group of lumber based on acoustic velocity(s) of lumber in the group.

16. A method of sorting pieces of green lumber into groups for drying comprising:
providing an impact on one end of a piece of lumber;
detecting vibrations in the piece of lumber caused by the impact;
determining the acoustic velocity of the piece of lumber from the detected vibrations and a length of the piece of lumber;
determining the modulus of elasticity of the piece of lumber from the determined acoustic velocity and a density of the piece of lumber; and
sorting the piece of lumber into one of the drying groups based on the modulus of elasticity of the lumber.

17. The method of claim 16, comprising:
assessing the attenuation of gamma rays passing through at least one area of the piece of lumber; and
determining the density of the piece of lumber from the attenuation of the gamma rays and a thickness of the piece of lumber.

18. The method of claim 17, comprising:
assessing the attenuation of gamma rays passing through two or more areas of the piece of lumber.

19. The method of claim 17, comprising:
measuring the thickness of the piece of lumber.

20. The method of claim 17, comprising:
cutting the piece of lumber to a known thickness before assessing the attenuation of gamma rays passing through at least one area of the piece of lumber.

21. The method of claim 16, wherein providing an impact on one end of the piece of lumber includes striking the one end of the piece of lumber with a striking device.

22. The method of claim 16, comprising:
detecting the presence of lumber; and
providing the impact on one end of the piece of lumber in response to detecting the presence of the piece of lumber.

23. The method of claim 16, wherein detecting vibrations in the piece of lumber includes detecting vibrations in the piece of lumber at one of the ends of the piece of lumber.

24. The method of claim 16, comprising:
digitally recording the vibrations detected in the piece of lumber; and
determining the acoustic velocity of the piece of lumber includes Fourier transforming the digitally recorded vibrations to determine the resonant frequency of the vibrations in the piece of lumber.

25. The method of claim 16, comprising:
determining the length of the piece of lumber.

26. The method of claim 16, comprising:
cutting the piece of lumber to a known length before providing the impact on one end of the piece of lumber.

27. The method of claim 16, comprising:
assessing a drying regime for each group of lumber based on modulus(s) of elasticity of lumber in the group.

28. The method of claim 16, wherein the drying is kiln drying.

29. A method of sorting green lumber conveyed on a conveyor system into groups for drying comprising:
assessing the attenuation of gamma rays passing through at least one area of a piece of lumber passing between a gamma ray source and detector;
determining the thickness of the piece of lumber as the piece of lumber passes thickness determining apparatus;
providing an impact on one end of the piece of lumber;
detecting vibrations in the piece of lumber caused by the impact as the piece of lumber passes a vibration sensor;
determining the length of the piece of lumber as the piece of lumber passes length determining apparatus;
determining the acoustic velocity of the piece of lumber from the detected vibrations and the length of the piece of lumber;
determining the density of the piece of lumber from the attenuation of the gamma rays and the thickness of the piece of lumber;
determining the modulus of elasticity of the piece of lumber from the determined acoustic velocity and the determined density of the piece of lumber; and
sorting the piece of lumber into one of the drying groups based on the modulus of elasticity of the lumber.

30. A system for sorting green lumber into groups for drying comprising
a striking device arranged to provide an impact on one end of the piece of lumber;
a vibration sensor arranged to detect vibrations in the piece of lumber caused by the impact on the piece of lumber by the striking device;
a computation device configured to determine the acoustic velocity of the piece of lumber from the detected vibrations and a length of the piece of lumber, the computation device being configured to determine the modulus of elasticity form the determined acoustic velocity and a density of the piece of lumber; and
an indication device for providing an indication of a one of the drying groups for the piece of lumber based on the determined modulus of elasticity.

31. The system of claim 30, comprising:
a gamma ray device arranged to pass gamma rays through at least one area of a piece of lumber and record the attenuation of the gamma rays passed through the piece of lumber;
wherein the computation device is further configured to determine the density of the piece of lumber from the attenuation of the gamma rays and a thickness of the piece of lumber.

32. The system of claim 31, wherein the gamma ray device is arranged to pass gamma rays through two or more areas of the piece of lumber and record the attenuation of the gamma rays passed through the piece of lumber.

33. The system of claim 31, comprising:
thickness measuring apparatus for measuring the thickness of the piece of lumber.

34. The system of claim 31, comprising:
cutting apparatus for cutting the piece of lumber to a known thickness before passing gamma rays through at least one area of a piece of lumber and recording the attenuation of the gamma rays passed through the piece of lumber.

35. The system of claim 30, comprising a device to detect the presence of the piece of lumber, the device to detect the presence of lumber being arranged to trigger operation of the striking device in response to detecting a piece of lumber.

36. The system of claim 30, wherein the vibration sensor is arranged to detect vibrations in the piece of lumber at one of the ends of the piece of lumber.

37. The system of claim 30, wherein the system is arranged to digitally record the detected vibrations, and the computation device is configured to Fourier transform the digitally recorded vibrations to determine the resonant frequency of the vibrations in the piece of lumber.

38. The system of claim 30, comprising:
length measuring apparatus for measuring the length of the piece of lumber.

39. The system of claim 30, comprising:
cutting apparatus for cutting the piece of lumber to a known length before the striking device provides the impact on one end of the piece of lumber.

40. The system of claim 30, wherein the computation device is configured to provide a drying regime for each group of lumber based on modulus(s) of elasticity of lumber in the group.
